(19) United States
(12) Patent Application Publication
(10) Pub. No.: US 2010/0101578 A1
(43) Pub. Date: Apr. 29, 2010

(54) ONE-WAY AIR FLOW VALVE USING ROTARY SPRING

(76) Inventors: Eun Jong Cha, Cheongiu-City (KR); Kyung Ah Kim, Cheongiu-City (KR); Wun Jae Kim, Seoul (KR)

Correspondence Address: GREER, BURNS & CRAIN, 300 S WACKER DR, 25TH FLOOR, CHICAGO, IL, 60606, US

(21) Appl. No.: 12/485,450

(22) Filed: Jun. 16, 2009

(30) Foreign Application Priority Data
Oct. 29, 2008 (KR) 10-2008-0106216

(51) Int. Cl. A61M 16/20; A61B 5/091

(52) U.S. Cl. 128/205.24; 600/540

(57) ABSTRACT

A one-way air flow valve includes one-way inspiration and expiration valve assemblies which have a same structure, are connected to each other in parallel, and are arranged opposite to each other, so that valve plates provided in the one-way inspiration and expiration valve assemblies, respectively, are open in directions opposite to each other, and a connection part including a mouthpiece provided at one side of the inspiration and expiration valve assemblies to allow an examinee to inhale or exhale air. One end of the inspiration valve assembly is connected with a gas tank, one end of the expiration valve assembly is connected with a gas density analyzer, and the inspiration and expiration valve assemblies include a valve and suction and discharging tubes coupled with one side and an opposite side of the valve, respectively.

### BACKGROUND OF THE INVENTION

1. Field of the Invention

The present invention relates to a one-way air flow valve. More particularly, the present invention relates to a one-way air flow valve, capable of preventing inspiration and expiration valves from being arbitrarily open even if valve stems do not maintain the horizontal state, capable of maintaining the inspiration and expiration valves in the closed state despite long-term use, and capable of facilitating replacement due to the separable structure thereof.

2. Description of the Prior Art

Generally, a one-way air flow valve is used to perform various clinical examinations by separating inspiration air from expiration air when humans breathe through their mouths. In other words, the one way-air flow valve is a medical tool essentially used for a clinical pulmonary function test, which measures the density of, for example, oxygen, carbon dioxide, and nitrogen by collecting expiration air resulting from physiological gas exchange of inhale air having specific composition (for example, 100% of oxygen)in the lugs.

The clinical pulmonary function test carried out through such a one way-air flow valve includes a single breath N₂ test measuring a closing volume, a multiple breath N₂ test measuring a functional residual capacity, and an exercise pulmonary function test measuring a metabolic rate.

As shown in FIG. 1, the one-way air flow valve according to the related art includes a body 10 having the form of a T-shaped tube. The body 10 is provided at both sides thereof with an inspiration port 12 and an expiration port 14. The inspiration port 12 receives air for inhalation of an examinee, and the expiration port 14 discharges air exhaled by the examinee. In addition, the body 10 is provided at the central portion thereof with a mouthpiece part 11 making contact with the mouth of the examinee. In this case, the inspiration port 12 and the expiration port 14 are provided therein with air valves 16 and 18 allowing air to exclusively flow in one direction.

The operational principles of the one-way air flow valve shown in FIG. 1 are as follows. First, when an examinee brings his/her mouth into contact with the mouthpiece part 11 and then tries to inhale air, a negative pressure is formed in the central portion of the body 10, so that the air valve 16 is open at the side of the inspiration port 12, and the air valve 18 is closed at the side of the expiration port 14. Since the valves 16 and 18 provided in the body 10 are one-way valves, the open/closed state of the valves 16 and 18 are determined according to a pressure applied thereto. After the examinee has inhaled air having specific composition through the open valve 16, when the examinee starts to exhale air, a positive pressure is formed at the central portion of the body, so that the valve 16 positioned at the inspiration port 12 is closed, and the valve 18 positioned at the expiration port 14 is open.

Accordingly, the expiration air is withdrawn from a tube through the open valve 18. The one-way air flow valve collects the expiration air withdrawn through the tube and analyzes the density of the air, thereby performing the clinical pulmonary function test.

As the one-way air flow valve operating based on the above operational principle, a two-way non-rebreathing valve (Hans-Rudolph, no 2700B, Kansas City, Mo., USA) is commercially available. This product includes the mouthpiece part 11 provided at the central portion of the body 10 having the form of a T-shaped tube to make contact with the mouth of the examinee, and the inspiration port 12 and the expiration port 14 provided at both sides of the body 10 as shown in FIG. 2, respectively.

The valves 16 and 18 provided in the inspiration port 12 and the expiration port 14, respectively, are manufactured by using a silicon plate S1 that may be open/closed. The silicon plate S1 is coupled with inner sides of the inspiration port 12 and the expiration port 14, in which silicon lines S2 are slantingly formed at the outer side of a coupling member S3 in a predetermined interval, and the silicon lines S2 are coupled with the silicon plate S1 having a circular shape.

In the one-way valves 16 and 18 having the above structure, when the same pressure is applied to both ends P0 and P1 of the valves as shown in FIG. 3, the silicon plate S1 is closed by the elasticity of the silicon lines S2. In this state, if the pressure of the left end P0 becomes higher than that of the right end P1 so that the silicon plate S1 overcomes the elasticity of the silicon lines S2, the silicon lines S2 are vertically open. Accordingly, since parallelogram spaces H are formed between the open silicon lines S2, air passes through the spaces H. Since such one-way valves 16 and 18 are formed in the inspiration port 12 and the expiration port 14, respectively, the valve 16 positioned at the inspiration port 12 is open when an examinee inhales air, and the valve 18 positioned at the expiration port 14 is open when the examinee exhales air.

However, since the conventional one-way air flow valve having the above structure must be open/closed by the elasticity of a silicon material of the valve, the silicon lines S2 must be exactly arranged. In addition, since it is difficult to manufacture the one-way air flow valve, the manufacturing cost is increased. Further, since the elasticity of silicon is degraded if the one-way air flow valve is repeatedly used several times, it is difficult to continuously maintain the one-way air flow valve in a closed state. Therefore, the one-way air flow valve is suitable for one-time use, so the usage cost may be increased.

After the clinical pulmonary function test has been performed using the one-way air flow valve, the whole part of the one-way air flow valve must be cleaned. However, if only the valve embedded in the one-way air flow valve is disposable, infection may not be controlled.

As shown in FIG. 3, since air must pass through a side surface of the valve in inspiration and expiration, the air flow is distorted. In addition, since the area of the open side surface has a limitation, an air circulation area is restricted. Accordingly, the breathing of the examinee may be interrupted.

In other words, since the one-way air flow valve has a T shape, and is provided at the central portion thereof with a mouthpiece tube allowing the breathing of the examinee, the flow of both inhaled and exhaled air is bent at an angle of 90□, so that the stream line of the air is distorted. Therefore, since the air must circulate along the stream line that is distorted, the examinee may have difficulty in breathing. In order to quantify respiratory disturbance caused by the structure of the valve and the tube having large respiration impedance, pressure variation is measured in the one-way air flow valve during the ordinary breathing of a normal adult through the mouthpiece tube formed at the central portion of the one-way air flow valve. The measured result shows the pressure variation of about 0.35 cmH2O that is a root-mean-square (RMS) value of a pressure signal as shown in FIG. 4. This means that the one-way air flow valve is able to be open/closed when an average value of the respiration pressure is at least 0.35 cmH2O in the mouthpiece tube formed at the central portion of the body of the one-way air flow valve. In addition, the result is measured by taking into consideration the respiration impedance that is the effect of the respiration disturbance of the examinee.

As a result, the conventional one-way air flow valve causes the respiration impedance disturbing the respiration of the examinee due to the structure thereof. As the respiration impedance is increased, the reliability of a clinical examination result may be degraded.

In order to solve the problem, Korean Patent No. 455450 has been issued by applicant of the subject invention.

The patent discloses a one-way air flow valve, which has the structure of a respiration tube capable of preventing the streamline of air flow from being disturbed, and is open/closed even in a low respiration pressure. This one-way air flow valve minimizes respiration impedance effect, that is, respiration disturbance, thereby extremely reducing the manufacturing cost and the maintenance cost.

As shown in FIG. 5, the one-way air flow value includes a first connection part 300, an expiration valve assembly 100, and an inspiration valve assembly 200. The first connection part 300 is provided at the central portion thereof with a mouthpiece coupling part 310 coupled with a mouthpiece 320 making contact with the mouth of an examinee. The expiration and inspiration valve assemblies 100 and 200 are connected with the first connection part 300 and provided therein with valve plates 130 and 230, which rotate about a rotational shaft 220 thereof to allowing air circulation, and is locked by a locking protrusion 240 to block air circulation. The expiration and inspiration valve assemblies 100 and 200 are connected to each other in parallel such that inlets are arranged in directions opposite to each other.

The second connection 400 is connected with an inlet and an outlet of the expiration valve assembly 100 and the inspiration valve assembly 200 and connected with a variety of appliances to separate inhaled air from exhaled air to perform a clinical pulmonary function test.

However, the conventional one-way air flow valve includes the valve plates 130 and 230 arranged in an oblique line. Accordingly, when performing the clinical pulmonary function test, an examiner must always maintain the valve plates 130 and 230 in a horizontal state. In other words, if the valve plates 130 and 230 are not in a horizontal state, an expiration valve may be inclined and open when an examinee inhales air, so that the reliability of the clinical pulmonary function test may be degraded.

In addition, since the valve plates 130 and 230 are slantingly sunk, the left and right parts of the valve assemblies 100 and 200 are excessively lengthened so that a respiration pressure may be reduced. Accordingly, the clinical pulmonary function test may not be reliably performed. In other words, if the length of the valve assemblies 100 and 200 is lengthened, since the internal volume is increased, inspiration and expiration pressures may be lowered. If the inspiration pressure is excessively lowered, a valve is unnecessarily open/closed due to the slight variation of external air pressure, so that the reliability of the clinical pulmonary function test may be degraded.

### SUMMARY OF THE INVENTION

Accordingly, the present invention has been made keeping in mind the above problems occurring in the prior art, and embodiments of the present invention provide a one-way air flow valve, capable of preventing a valve plate from arbitrarily being open by the elasticity of a spring in an upright state and adjustably increasing inspiration and expiration pressures because the moving angle of the valve plate is narrow.

According to one aspect of the present invention, a one-way air flow valve includes one-way inspiration and expiration valve assemblies which have a same structure, are connected to each other in parallel, and are arranged opposite to each other, so that valve plates provided in the one-way inspiration and expiration valve assemblies, respectively, are open in directions opposite to each other, and a connection part including a mouthpiece provided at one side of the inspiration and expiration valve assemblies to allow an examinee to inhale or exhale air, wherein one end of the inspiration valve assembly is connected with a gas tank, one end of the expiration valve assembly is connected with a gas density analyzer, the inspiration and expiration valve assemblies comprise a valve and suction and discharging tubes coupled with one side and an opposite side of the valve, respectively, the valve is provided therein with an input/output hole, a sealing plate is attached around the input/output hole, and one side of the input/output hole is hinged with a valve plate, to which an O-ring making contact with the sealing plate is attached, at an upper portion of the valve plate such that the valve plate is elastically rotated by a coil spring.

A locking protrusion is formed at an outer central portion of the valves to fix positions of the suction and discharging tubes and a position of the connection tube.

The valve plate is integrally formed with a hinge plate, through which a hinge shaft passes, at an upper end of the valve plate, one end of the coil spring is seated on an upper portion of the valves, and an opposite end of the coil spring is fixedly inserted in a groove formed in one side surface of the valve plate.

As described above, in the one-way air flow valve according to the present invention, even if an examiner examines an examinee while rotating the one-way air flow valve according to the position of the examinee, the examiner can obtain exact data due to the close contact of the valve plate by a spring.

In addition, the length of the inspiration and expiration valve assemblies is reduced as much as possible, thereby increasing inspiration and expiration pressures, so that the reliability of the examination for the examinee can be enhanced.

Further, since only the valve plate can be separated from the one-way air flow valve, replacement and maintenance works for the one-way air flow valve can be easy.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a view showing the operation procedure of a one-way air flow valve according to the related art;

FIG. 2 is an exploded perspective view showing the structure of the one-way air flow valve according to the related art;

FIG. 3 is a view showing the operation of a silicon valve used in the one-way air flow valve according to the related art;

FIG. 4 is a graph showing a respiration disturbance effect of the silicon valve of FIG. 3;

FIG. 5 is a perspective view showing an improved one-way air flow valve capable of minimizing the respiration disturbance effect;

FIG. 6 is a perspective view showing a one-way air flow valve according to a first embodiment of the present invention;

FIG. 7 is an exploded perspective view showing an inspiration valve assembly of FIG. 6;

FIG. 8 is a sectional view longitudinally showing a valve of FIG. 7;

FIG. 9 is a view showing the operation of the one-way air flow valve according to the first embodiment of the present invention when a pulmonary function test is performed;

FIG. 10 is a perspective view showing a one-way air flow valve according to a second embodiment of the present invention; and

FIG. 11 is a view showing the operation of the one-way air flow valve according to the second embodiment of the present invention when a pulmonary function test is performed.

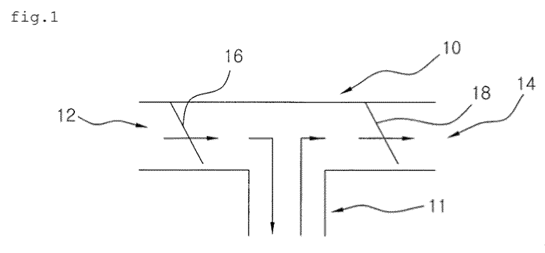

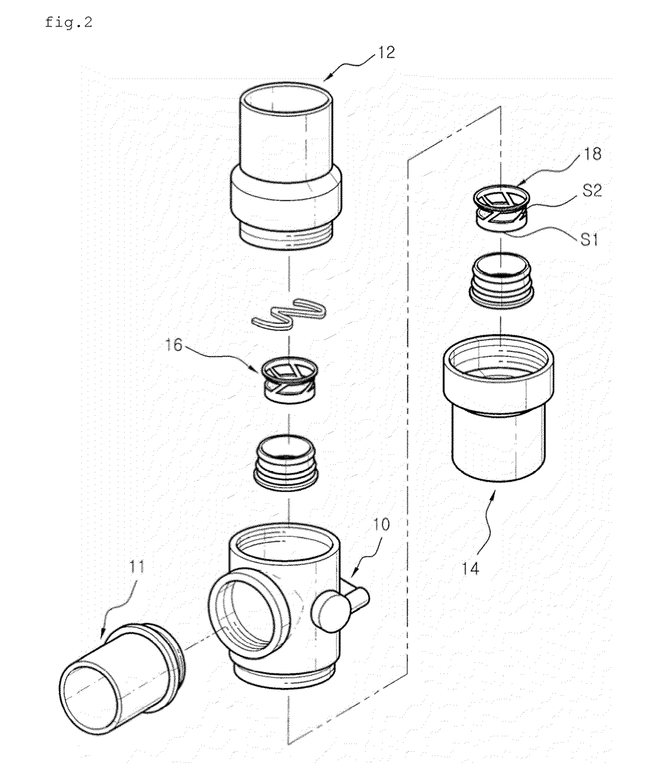

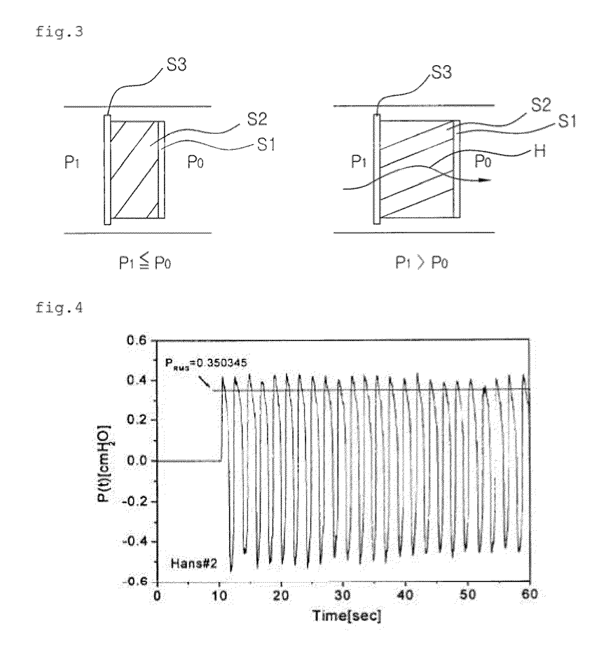

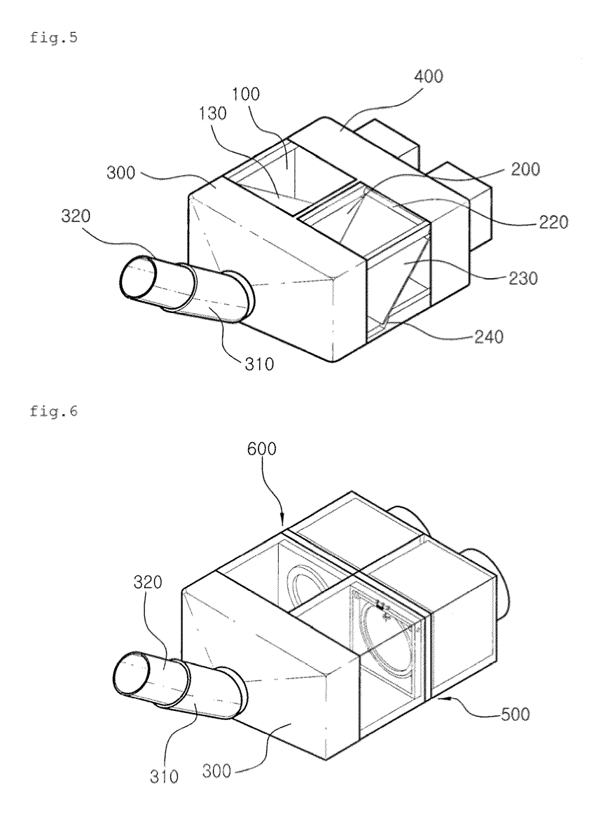

### DETAILED DESCRIPTION OF THE INVENTION

Reference will now be made in greater detail to exemplary embodiments of the invention with reference to the accompanying drawings.

Hereinafter, a one-way air flow valve employing a rotary spring according to a preferred embodiment of the present invention will be described with reference to accompanying drawings.

### Embodiment 1

FIG. 6 is an assembled perspective view showing a one way air flow valve according to a first embodiment of the present invention.

Referring to FIG. 6, the one way air flow valve according to the present invention has a structure in which an inspiration valve assembly 500 and an expiration valve assembly 600 are connected to each other in parallel.

A connection part 300 is formed at one side of the inspiration valve assembly 500 and the expiration valve assembly 600 that are connected to each other in parallel. The connection part 300 includes a rubber material and is provided at a central portion thereof with a mouthpiece coupling part 310, which receives a mouthpiece 320 made of a paper material and has a tube shape.

The inspiration valve assembly 500 and the expiration valve assembly 600 have the same shape and the same function, but the orientation of the inspiration valve assembly 500 is opposite to that of the expiration valve assembly 600.

Accordingly, only the inspiration valve assembly 500 will be representatively described below.

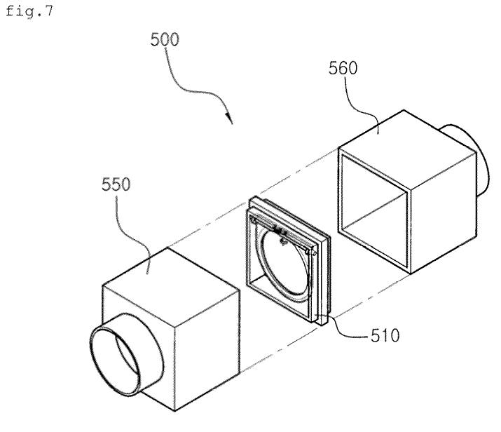

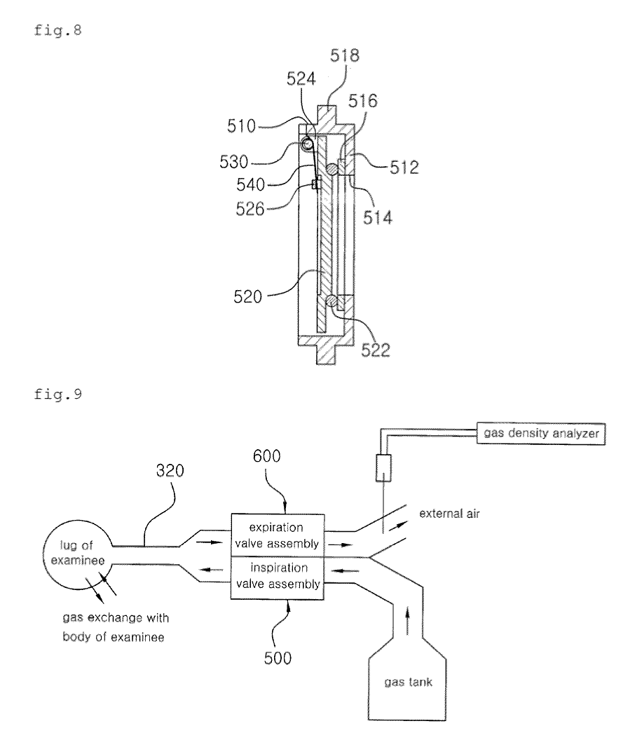

FIG. 7 is an exploded perspective view showing the inspiration valve assembly 500 of FIG. 6, and FIG. 8 is a sectional view showing longitudinally the inspiration valve assembly 500 of FIG. 7.

Referring to FIGS. 7 and 8, the inspiration valve assembly 500 includes a valve 510 opening/closing a valve plate 520 in one direction, and the valve 510 is provided at one side thereof with a discharge tube 550 and the other side thereof with a suction tube 560.

The valve 510 has a rectangular body having a short length. A support plate 512 formed with an input/output hole 514 having a relatively small size is integrally formed with the other side of the valve 510. A sealing plate 516 including a silicon material is attached onto one side of the support plate 512 around the input/output hole 514.

Meanwhile, the valve plate 420 having a circular shape is hinged to an upper portion of an inner side of the valve 510 such that the valve plate 420 rotates. In detail, a hinge plate 524 is integrally formed with the upper surface of the valve plate 520 while protruding in one direction from the upper surface of the valve 520. In addition, a hinge shaft 530 is provided at one side of the inner part of the valve 510 and fixed onto both walls of the valve 510 while passing through the hinge plate 524.

A coil spring 540 having two opposite ends is fitted around a central portion of the hinge shaft 530 such that one end of the coil spring 540 is seated on the upper portion of the valve 510 and the other end of the coil spring 540 is seated on one surface of the valve plate 520. A groove 526 is formed on the surface of the valve plate 520 such that the other end of the coil spring 540 is restrained.

In addition, O-rings 522 are attached to an opposite surface of the valve plate 520 such that the O-rings 522 closely make contact with the sealing plate 516, thereby blocking air flow when the valve plate 520 is in an upright position.

Further, the valve 510 is provided at an outer central portion thereof with locking protrusions 518, which radially protrude from the outer central portion such that the suction tube 560 and the discharge tub 550 are fixed to the valve 510 when the suction tube 560 and the discharge tub 550 are fitted to the valve 510.

FIG. 9 is a view showing the operational procedure for a pulmonary function test in one way air flow valve according to the present invention.

As shown in FIG. 9, since the suction tube 560 is connected with a gas tank, and the discharge tube 550 is connected with the mouthpiece 320, the inspiration valve assembly 500 having the above structure serves as a passage of gas that an examinee inhales.

In contrary, the expiration valve assembly 600 serves as a passage of gas that the examinee exhales. The expiration valve assembly 600 is disposed in opposition to the inspiration valve assembly 500, so that a tube provided at the side of the mouthpiece 320 serves as the suction tube 560, and an opposite tube serves as a discharge tube 550.

### Embodiment 2

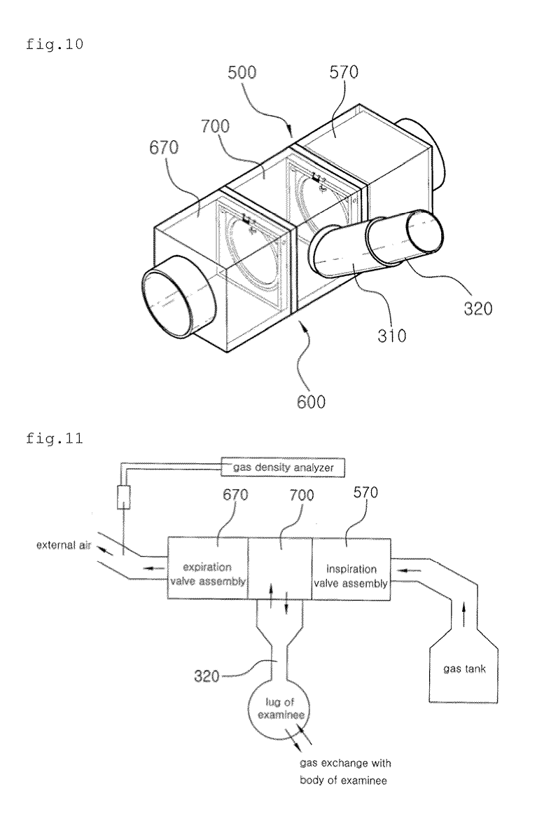

FIG. 10 is an assembled perspective view showing a one way air flow valve according to a second embodiment of the present invention, and FIG. 11 is a view showing the operational procedure for a pulmonary function test in the one way air flow valve according to the second embodiment of the present invention.

Referring to FIG. 10, the one way air flow valve according to the second embodiment of the present invention includes the inspiration valve assembly 500 and the expiration valve assembly 600, which are identical to those of the first embodiment, but they are connected to each other in series while being aligned in the same direction.

The inspiration valve assembly 500 is connected with the expiration valve assembly 600 through a connection tube 700, and the connection tube 700 is provided with the mouthpiece 320 similarly to the first embodiment.

The suction tube 570 is coupled to one side of the connection tube 700 of the inspiration valve assembly 500 for inspiration, and the discharge tube 670 is coupled to an opposite side of the connection tube 700 for expiration.

When comparing with a T-shape air flow valve according to the related art, the one way air flow valve according to the second embodiment of the present invention is modified only in the structure of the valve assembly, and provides the same air flow.

Accordingly, the suction tube 570 is connected to a gas tank as shown in FIG. 11 such that air is introduced into the inspiration valve assembly 500 when the examinee inhales through the mouthpiece 320. The discharge tube 670 is connected to a gas density analyzer such that the air is discharged through the expiration valve assembly 600 when the examinee exhales.

Although an exemplary embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A one-way air flow valve comprising:
one-way inspiration and expiration valve assemblies which have a same structure, are connected to each other in parallel, and are arranged opposite to each other, so that valve plates provided in the one-way inspiration and expiration valve assemblies, respectively, are open in directions opposite to each other; and
a connection part including a mouthpiece provided at one side of the inspiration and expiration valve assemblies to allow an examinee to inhale or exhale air,
wherein one end of the inspiration valve assembly is connected with a gas tank, one end of the expiration valve assembly is connected with a gas density analyzer, the inspiration and expiration valve assemblies comprise a valve and suction and discharging tubes coupled with one side and an opposite side of the valve, respectively, the valve is provided therein with an input/output hole, a sealing plate is attached around the input/output hole, and one side of the input/output hole is hinged with a valve plate, to which an O-ring making contact with the sealing plate is attached, at an upper portion of the valve plate such that the valve plate is elastically rotated by a coil spring.

2. A one-way air flow valve comprising:
inspiration and expiration valve assemblies, which have a same structure and are coupled to both open sides of a connection tube having a mouthpiece, respectively, such that a valve plate is open in the same direction, the inspiration valve assembly being connected with a gas tank, the expiration valve assembly being connected with a gas analyzer,
wherein the inspiration and expiration valve assemblies include valves connected with both sides of the connection tube, and suction and discharging tubes coupled with opposite sides of the valves, each valve is formed therein with an input/output hole, a sealing plate is attached around the input/output hole, and one side of the input/output hole is hinged with a valve plate, to which an O-ring making contact with the sealing plate is attached, at an upper portion of the valve plate such that the valve plate is elastically rotated by a coil spring.

3. The one-way air flow valve of claim 1, wherein a locking protrusion is formed at an outer central portion of the valves to fix positions of the suction and discharging tubes and a position of the connection tube.

4. The one-way air flow valve of claim 1, wherein the valve plate is integrally formed with a hinge plate, through which a hinge shaft passes, at an upper end of the valve plate, one end of the coil spring is seated on an upper portion of the valves, and an opposite end of the coil spring is fixedly inserted in a groove formed in one side surface of the valve plate.
